(19) United States
(12) Patent Application Publication
(10) Pub. No.: US 2014/0147406 A1
(43) Pub. Date: May 29, 2014

(54) HAIR VOLUMIZING COMPOSITIONS AND METHODS

(71) Applicant: Living Proof, Inc., Cambridge, MA (US)

(72) Inventors: Ronald P. McLaughlin, Reading, MA (US); Maria Moga, Medford, MA (US)

(73) Assignee: Living Proof, Inc., Cambridge, MA (US)

(21) Appl. No.: 14/168,232

(22) Filed: Jan. 30, 2014

Related U.S. Application Data
(62) Division of application No. 12/610,786, filed on Nov. 2, 2009.

(51) Int. Cl. A61K 8/81; A61Q 5/06; A61K 8/72

(52) U.S. Cl. CPC A61K 8/8152; A61K 8/8111; A61K 8/72; A61Q 5/06
USPC 424/70.11

(57) ABSTRACT

Compositions for treating hair that provide volumizing and stylability to hair.

### BACKGROUND OF THE INVENTION

This application is a divisional of U.S. application Ser. No. 12/610,786, filed Nov. 2, 2009, which is incorporated by reference herein in its entirety.

### FIELD OF THE INVENTION

The invention is directed to compositions and methods for treating hair, and in particular to compositions and methods for imparting thickness, volume, texture and definition, which permit improved styling.

### DESCRIPTION OF RELATED ART

People with fine or thin hair regularly use “volumizing” products in the form of shampoos, conditioners, styling sprays, creams, gels, or mousses in an effort to increase the volume and body of their hair. Many of these products utilize film forming resins which bind multiple hair fibers together. The products form a film of the product on a section of the hair, linking a hair fiber back on itself and/or linking two or more fibers, creating stiffer hair, more resistant to falling flat on the head.

It has been discovered that certain commercially available compounds, some of which are rubbers, rubber like compounds, adhesives or having adhesive-like properties may be formulated in a hair care composition and used in methods to increase thickness, volume and texture and to impart stylability to scalp hair.

It is desired to identify and develop compositions that achieve the abovementioned results through the use of cosmetically acceptable components not conventionally used for this purpose.

### SUMMARY OF THE INVENTION

In one aspect, the invention is a hair treatment composition comprising a rosin selected from the group consisting of diethylene glycol rosinate; dimer dilinoleyl hydrogenated rosinate; glycol rosinate; pentaerythrityl rosinate; and triethylene glycol rosinate, in a form that can be applied to hair.

In another aspect, the invention is a hair treatment composition comprising a rosin or modified rosin selected from the group consisting of dimerized gum rosin, partially dimerized gum rosin; and partially hydrogenated gum rosin, in a form that can be applied to hair.

In another aspect, the invention is a hair styling composition comprising a glycerol ester, a synthetic resin sold under the trade name Ester Gum 8D, Ester Gum 8D-C, Ester Gum 8D-M, Ester Gum 8D-M, Kosher, Ester Gum 8L-M Resin, Ester Gum 8WA-M, Ester Gum 8WA-M, Kosher, or Ester Gum 15D-M, Ester Gum 10-D, or a glyceryl hydrogenated rosinate; hydrogenated gum rosin; ester of hydrogenated rosin known under the trade names Foral 85-E; Staybelite Ester 5-E; Foral 86-E FG; Staybelite Ester 5-C; Foralyn 90; Staybelite Ester 10-E; Foralyn 90-FG; Permalyn 2085 or 5095-C. These active ingredients are referred to herein as “ester gums”.

In another aspect, the invention is a hair styling composition comprising a methyl ester selected from the group consisting of methyl ester of rosin; methyl hydrogenated rosinate; or a rosin sold under the Trade Name Metalyn 200.

In another aspect, the invention is a hair styling composition comprising a pentaerythritol ester selected from the group consisting of mixed glycerine-pentaerythritol ester of gum rosin; ester of hydrogenated rosin; pentaerythrityl tetraabietate, pentaerythrityl hydrogenated rosinate, hydrogenated gum rosin, or various other synthetic resins sold under the trade names Pentalyn C, Pentalyn H-E, Pentalyn 511-M, and Permalyn 3100, 5110, 5110-C and 6110.

In another aspect, the invention is a hair styling composition comprising a triethylene glycol hydrogenated rosinate, such as that sold under the tradename Staybelite Ester 3-E.

In another aspect, the invention is a hair styling composition comprising a synthetic resin, such as that sold under the tradename Cellolyn 21-E; or a modified ester selected from the group consisting of maleic acid modified glycerol ester of gum rosin; gum rosin-maleic acid modified pentaerythritol ester; gum rosin-fumaric acid modified pentaerythritol ester; and phenolic modified pentaerythritol ester, such as are variously sold under the trade names Pentalyn 601-M, 615-M, 670-M, 702-M, 711-M, and 750-HV-M.

In another aspect, the invention is a hair styling composition comprising a sodium or potassium rosin soap, such as sold under the tradename Dresinate TX or Dresinate 91.

In another aspect, the invention is a hair styling composition comprising an adhesion promoter selected from the group consisting of sucrose acetate isobutyrate; sucrose acetate isobutyrate in alcohol medium; sucrose acetate isobutyrate in caprylic/capric triglyceride medium; and sucrose acetate isobutyrate in ethyl acetate medium.

In another aspect, the invention is a hair styling composition comprising a transdermal pressure sensitive active ingredient selected from the group consisting of acrylate-vinylacetate; acrylate; acrylic-rubber hybrid; acrylic-rubber hybrid tackifier; polyisobutylene; and styrenic rubber tackifier.

In another aspect, the invention is a hair styling composition comprising a rubber latex or rubber latex extract.

In another aspect, the invention is a hair styling composition comprising a diene-containing copolymer selected from the group consisting of butadiene/acrylonitrile copolymer; dicyclopentadiene/isopentene/isoprene copolymer dicyclopentadiene/isopentene/isoprene/styrene copolymer; hydrogenated dicyclopentadiene/isopentene/isoprene copolymer; hydrogenated dicyclopentadiene/isopentene/isoprene/styrene copolymer; piperylene/butene/pentene/pentadienecopolymer; polycyclopentadiene; and sodium PVM/MA/decadiene crosspolymer.

In another aspect, the invention is a hair styling composition comprising a “prene” compound selected from the group consisting of isobutylene/isoprene copolymer; and styrene/isoprene copolymer.

In another aspect, the invention is a hair styling composition comprising a polyvinyl acetate, such as sold under the tradename UCAR 130 Latex Resin.

In another aspect, the invention is a method of treating hair, to provide volume or styling the hair, comprising the step of applying at least one of the aforesaid hair treatment compositions to scalp hair in an amount effective to impart volume or stylability to hair.

At least some of the abovementioned composition components have been used in cosmetics, but not in a composition for treatment of scalp hair. It is believed that the abovementioned ingredients, alone or formulated in a hair care composition, exhibit elastic and/or adhesive properties under shear to provide the desired functional attributes in a hair treatment composition. The elastic and/or adhesive properties may be assessed using test procedures described herein.

### DETAILED DESCRIPTION OF THE PREFERRED EMBODIMENTS

The compositions according to the invention achieve volume and texture enhancement, provide thickness and impart stylability using ingredient that have been used in the cosmetics industry, but not in the context of hair care. In many cases, the compositions according to the invention utilize industrial commercial materials which have not been used in the cosmetic arts at all.

The compounds described herein for use in hair are generally commercially available. Where a compound is identified by trade name, it is understood to refer to the compound available from the supplier associated with that trade name as of the filing date of this application. Thus, for example, deiethylene glycol rosinate is available from Arizona Chemical (Jacksonville, Fla.) under the trade name Sylvatec RE 25, and pentaerythrityl rosinate is available from Hercules Chemical Company Inc. (Passaic, N.J.) under the trade name Pentalyn A. However, these generic compounds may be available from other suppliers, and the use of the generic name is intended to refer to the compound from any source. If a component is identified only by its trade name, it is intended to refer to the specific component available from that supplier.

Many of the components used in the compositions according to the inventions are resins. “Resin” as used herein refers to a hardenable organic substance typically associated with a plant secretion, such as pine sap, which may be found in nature or produced synthetically (synthetic resin). Rosins and rosinates are also resins—and these words often refer to a processed resin. Ester Gum, Dymerex, Foral, Staybelite, Foralyn, Abalyn, Metalyn, Poly-Pale, Pentalyn, Cellolyn, Lewisol, Dresinate, and Sustaine all refer to natural and synthetic resins and rosins sold by Eastman Chemical Co., Kingsport, Tenn. under those trade names. These trade names are usually associated with a further alphanumeric identifier.

Polymeric materials which may be used as active ingredients in the compositions of the invention are available from many sources. For example, styrene/isoprene copolymer is available from Shell Chemical Company under the trade name Kraton D-1161 NS.

Transdermal pressure sensitive adhesives which can be used as an active ingredient in a composition according to the invention include: isobutylene, commercially available from National Starch under the trade name Duro-Tak 87-608A; and acrylate-vinylacetates, available under the trade names Duro-Tak 387-2052 and Duro-Tak 87-4098.

An “active ingredient” in a hair treatment composition according to the invention is an ingredient which is provided in an amount effective to provide a volumizing or other styling effect when applied to hair. The active ingredient may in some instances be in a form that can be applied “neat,” directly on hair, but more often the active ingredient is formulated with a cosmetically acceptable excipient. The active ingredient may be provided in an amount of 0.01 percent by weight to 100 percent by weight of the composition. The optimal amount of active ingredient will depend on the application and the nature of the active ingredient. Typically the active ingredient is present in an amount of 0.1 percent by weight to 25 weight percent of the composition.

The compositions of the invention may be formulated with any cosmetically acceptable excipient that provides for application of the composition to scalp hair, and may be in the form of a spray, cream, lotion, emulsion, gel, wax, or pomade, for example. In one embodiment, a composition according to the invention is a styling cream, which may be left in the scalp hair after application without rinsing. In another embodiment, a composition according to the invention is a spray.

The compositions according to the invention may be water-based or water-alcohol based, or solvent-based. Water based means that a composition contains greater than 50 percent by weight water, preferably greater than 70 percent by weight water, and more preferably greater than 80 percent by weight water. Likewise, solvent-based means that an organic solvent is present in the composition in an amount greater than 50 percent by weight, preferably greater than 70 percent by weight, and more preferably greater than 80 percent by weight. Organic solvents that may be used in the compositions of this invention are well know to those skilled in the art.

A cosmetically acceptable carrier for the compound contains ingredients conventionally used in hair treatment compositions. One or more emulsifiers may be included in a range of about 0.1 to about 10.0 percent by weight, in preferred embodiments, in a range of about 0.5 percent by weight to about 3.0 percent by weight. Suitable formulation amounts may be determined by those of ordinary skill in the art without undue experimentation, depending on how the composition is intended to be delivered. Suitable emulsifier systems may be selected from those known in the art, including without limitation, Polysorbate 20, Polyacrylate-13, Polyisobutene, Oleth-10, and the like.

A fatty alcohol, or a mixture, may be provided in an amount of 0.1 to about 5.0 percent by weight of the composition to provide thickening and/or emollient properties. In embodiments, a fatty alcohol component such as cetearyl alcohol (a mixture of fatty alcohols), may be provided in a range of about 0.1 to 1.0 percent by weight of the composition.

One or more fatty acids may be provided in a range of about 0.1 percent by weight to 5.0 percent by weight of the composition. Fatty acids, include without limitation, palmitic acid, stearic acid, linoleic acid, myristic acid, and others known in the art to provide thickening and/or emollient properties in a hair treatment composition.

Perfumes, stabilizers, preservatives, emollients, protectants, UV-protective agents, and other ingredient conventionally found in hair care products may be provided in customary amounts, typically up to about 5 percent by weight of the composition.

### Formulation Example 1-1

A styling cream according to the invention may include the following:

| Ingredients | % w/w |
|---|---|
| Water | q.s. |
| Active ingredient | 0.50-6.00 |
| Polysorbate 20 (and) Polyacrylate-13 (and) Polyisobutene | 0.50-4.00 |
| (Emulsifier) | |
| Fragrance | 0.20-3.00 |
| Phenoxyethanol (and) Methylisothiazolinone (Preservative) | 0.50-1.50 |
| PPG-2 Myristyl Ether Propionate (Emollient) | 0.10-5.00 |
| Oleth-10 (Emulsifier) | 0.10-2.00 |
| Myristyl Alcohol (Co-Emulsifier) | 0.10-2.00 |
| Xanthan Gum (Rheology modifier) | 0.10-1.50 |

### Formulation Example 1-2

A styling spray according to the invention may include the following:

| Ingredients | % w/w |
|---|---|
| Water | q.s. |
| Active ingredient | 0.50-6.00 |
| Oleth-10 (Emulsifier) | 0.10-10.00 |
| Ethoxydiglycol Oleate (Emollient) | 0.10-5.00 |
| Fragrance | 0.20-3.00 |
| Phenoxyethanol (and) Methylisothiazolinone (Preservative) | 0.50-1.50 |
| Sodium Chloride | 0.10-1.50 |
| Xanthan Gum (Rheology modifier) | 0.10-1.50 |

Styling creams were formulated according to Formulation Example 1-1 having 3.0 grams, in each chase, of the active ingredients identified in Table 1.

TABLE 1

| Trade Name | Product Class |
|---|---|
| Duro-Tak 87-608A | Polyisobutylene (Transdermal Pressure |
| | Sensitive Adhesive) |
| Foral 85-E | Glyceryl Hydrogenated Rosinate |
| | (Glycerol Ester) |
| Duro-Tak 387-2052 | Acrylate-vinylacetate (Transdermal |
| | Pressure Sensitive Adhesive) |
| Staybelite Ester 5-E | Glyceryl Hydrogenated Rosinate (Glycerol |
| | Ester) |
| Duro-Tak 87-2074 | Acrylate (Transdermal Pressure Sensitive |
| | Adhesive) |
| Duro-Tak 87-4098 | Acrylate-vinylacetate (Transdermal |
| | Pressure Sensitive Adhesive) |
| DuroTak 87-9088 | Acrylate (Transdermal Pressure Sensitive |
| | Adhesive) |

### Friction Properties

In the most preferred embodiments, compositions according to the invention provide increased friction to the hair. To evaluate the frictional properties of hair treated with the formulations, four of the formulations containing transdermal pressure sensitive adhesives were selected. None of the four active ingredients in these formulations have heretofore been used in hair styling composition. Hair tresses were washed, under faucet, with a commercially available shampoo. Tresses were then rinsed until all shampoo was removed; excess water was removed by pressing between fingers. Tresses were mounted on a faux head with pins. All tresses were then combed through to ensure no knots or tangles. 0.8 grams of styling cream was applied to each tress. Tresses were styled using a round brush and a hair dryer on high heat.

The tresses were blinded, and a panel of four participants was assembled and asked to feel the hair, dragging the tress along the fingers and also pinching strands together, so that both grip and drag aspects of the frictional properties were evaluated together. The panel participants rated each of the formulations from 1 to 5, with 1 representing the most frictional properties and 5 representing the least. The results are tabulated in Table 2.

TABLE 2

| Trade Name | Rating |
|---|---|
| Duro-Tak 87-608A | 1.5 |
| Duro-Tak 87-2074 | 3.25 |
| Duro-Tak 87-4098 | 3.75 |
| DuroTak 87-9088 | 4.25 |

Qualitative assessments (but not side by side ratings) were made for all of the tresses treated, with the results shown in Table 3.

TABLE 3

| Trade Name | Comments |
|---|---|
| Duro-Tak 87-608A | Good friction |
| Foral 85-E | Good friction |
| Duro-Tak 387-2052 | Good friction |
| Staybelite Ester 5-E | Good friction; too smooth |
| Duro-Tak 87-2074 | Minimal grip/friction |
| Duro-Tak 87-4098 | No friction - too smooth |
| DuroTak 87-9088 | No Friction |

A formulation that received a comment of “good friction” is deemed to be according to the invention. Therefore, the fifth through seventh entries in Table 3 are comparative examples.

Notwithstanding anything herein to the contrary, the foregoing embodiments are exemplary only and not to be considered as limiting the invention, which is defined by the appended claims.

1-6. (canceled)

7. A method for treating scalp hair comprising: applying to scalp hair a composition comprising a rosin active ingredient selected from the group consisting of diethylene glycol rosinate; dimer dilinoleyl hydrogenated rosinate; glycol rosinate; pentaerythrityl rosinate; and triethylene glycol rosinate, in a form that can be applied to hair, in an amount effective to increase the volume of the hair; or
a modified rosin active ingredient selected from the group consisting of dimerized gum rosin; partially hydrogenated gum rosin; and partially dimerized gum rosin, in a form that can be applied to hair, in an amount effective to increase the volume of the hair.
applying to scalp hair a composition comprising an ester gum active ingredient, in a form that can be applied to hair, in an amount effective to increase the volume of the hair; or
applying to scalp hair a composition comprising a methyl ester active ingredient selected from the group consisting of methyl ester of rosin and methyl hydrogenated rosinate, in a form that can be applied to hair, in an amount effective to increase the volume of the hair; or
applying to scalp hair a composition comprising a pentaerythritol ester active ingredient selected from the group consisting of mixed glycerine-pentaerythritol ester of gum rosin; ester of hydrogenated rosin; pentaerythrityl tetraabietate; pentaerythrityl hydrogenated rosinate; and hydrogenated gum rosin, in a form that can be applied to hair, in an amount effective to increase the volume of the hair; or
applying to scalp hair a composition comprising a triethylene glycol hydrogenated rosinate active ingredient, in a form that can be applied to hair, in an amount effective to increase the volume of the hair; or
applying to scalp hair a composition comprising a modified ester active ingredient selected from the group consisting of synthetic resin 9; maleic acid modified glycerol ester of gum rosin; gum rosin-maleic acid modified pentaerythritol ester; gum rosin-fumaric acid modified pentaerythritol ester; and phenolic modified pentaerythritol ester, in a form that can be applied to hair, in an amount effective to increase the volume of the hair, in an amount effective to increase the volume of the hair; or
applying to scalp hair a composition comprising a sodium or potassium rosin soap active ingredient, in a form that can be applied to hair, in an amount effective to increase the volume of the hair; or
applying to scalp hair a composition comprising an active ingredient selected from the group consisting of sucrose acetate isobutyrate; sucrose acetate isobutyrate in alcohol medium; sucrose acetate isobutyrate in caprylic/capric triglyceride medium; and sucrose acetate isobutyrate in ethyl acetate medium, in a form that can be applied to hair, in an amount effective to increase the volume of the hair; or
applying to scalp hair a composition comprising a transdermal pressure sensitive active ingredient selected from the group consisting of acrylate-vinylacetate; acrylate; acrylic-rubber hybrid; acrylic-rubber hybrid tackifier; polyisobutylene; and styrenic rubber tackifier, in a form that can be applied to hair, in an amount effective to increase the volume of the hair; or
applying to scalp hair a composition comprising a rubber latex or rubber latex extract active ingredient, in a form that can be applied to hair, in an amount effective to increase the volume of the hair; or
applying to scalp hair a composition comprising a diene-containing copolymer active ingredient selected from the group consisting of butadiene/acrylonitrile copolymer; dicyclopentadiene/isopentene/isoprene copolymer dicyclopentadiene/isopentene/isoprene/styrene copolymer; hydrogenated dicyclopentadiene/isopentene/isoprene copolymer; hydrogenated dicyclopentadiene/isopentene/isoprene/styrene copolymer; piperylene/butene/pentene/pentadienecopolymer; polycyclopentadiene; and sodium PVM/MA/decadiene crosspolymer, in a form that can be applied to hair, in an amount effective to increase the volume of the hair; or
applying to scalp hair a composition comprising a prene-containing copolymer active ingredient selected from the group consisting of isobutylene/isoprene copolymer and styrene/isoprene copolymer, in a form that can be applied to hair, in an amount effective to increase the volume of the hair; or
applying to scalp hair a composition comprising a polyvinyl acetate active ingredient, in a form that can be applied to hair, in an amount effective to increase the volume of the hair.
